(19) United States
(12) Patent Application Publication
(10) Pub. No.: US 2019/0032067 A1
(43) Pub. Date: Jan. 31, 2019

(54) AUTOMATED METHODS AND SYSTEMS FOR GENETICALLY ENHANCING GENOMES IN COMPUTER CONTROLLED ENVIRONMENTS UTILIZING RECURRENTLY SEGMENTED NUCLEOTIDE REPLICATIONS

(71) Applicant: Organic Genomics, Inc., Peninsula, OH (US)

(72) Inventor: Timothy Madden, Peninsula, OH (US)

(73) Assignee: Organic Genomics, Inc., Peninsula, OH (US)

(21) Appl. No.: 16/049,111

(22) Filed: Jul. 30, 2018

Related U.S. Application Data
(60) Provisional application No. 62/538,430, filed on Jul. 28, 2017.

(51) Int. Cl. C12N 15/82; C12N 15/10

(52) U.S. Cl. CPC C12N 15/8216; C12N 15/1089; C12N 15/1082; C12N 15/1068

(57) ABSTRACT

One or more techniques, devices and systems described herein can be used for propagating cloned genes sequences for plant characteristic enhancement, and/or to generate plant material with enhanced characteristics. For example, automated systems for gene sequence propagation may be devised, and fewer resources for plant tissue culture/propagation, plant transformation, genetic engineering, and digital cloning than prior techniques can be used. The systems, devices and methods described herein can provide an automated and autonomous system, including a controlled environment comprising controls for temperature, humidity, light, and hygiene, to improve gene sequence culturing, cloning and harvesting, and for growth of plant cells or plantlets propagation into whole plants.

### CROSS-REFERENCE TO RELATED APPLICATIONS

This application claims priority to U.S. Provisional Patent Application Ser. No. 62/538,430, entitled AUTOMATED METHODS AND SYSTEMS FOR GENETICALLY MODIFIED PLANT CELL OR PLANTLET GROWTH INTO WHOLE PLANTS IN COMPUTER CONTROLLED ENVIRONMENTS, filed Jul. 28, 2017; which is incorporated herein in its entirety by reference.

### BACKGROUND

Plant breeding or propagation uses many techniques such cross pollinations, marker assisted selection, cloning, plant transformations or genetic medications to develop distinct varieties of whole plant crops. These specialty biological techniques require highly skilled scientists or technicians and laboratory facilities with critical horticulture and environmental equipment to perform these critical steps for successful production of viable, healthy plant stock. Advancements in technology on all fronts are evident these days. The goal of developing any new technology is to make the process easier and time efficient. Similar advancements had been made in the field of plant breeding and plant biotechnology. For example, sequencing the genome of a plant species can currently be performed in hours rather than the years that it formerly took to perform genome sequencing.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

One or more techniques, devices and systems described herein can be used for propagating cloned genes sequences for plant characteristic enhancement, and/or to generate plant material with enhanced characteristics. As an example, automated systems can be used for target gene sequence propagation, resulting in cloned DNA segments comprising desired characteristics. Further, the target gene sequences and autonomously be implanted in target plant tissue, and autonomously cultured to mature plant matter. These techniques and systems may utilize fewer resources for gene sequence cloning and/or plant tissue culture/propagation, plant transformation, genetic engineering, and digital cloning than prior techniques.

In one implementation of system for cloned genes sequences for plant characteristic enhancement a gene sequence creator can be used to autonomously create a fragment gene sequence that comprise a desired characteristic expression from a target organism, from deoxynucleic acid (DNA) obtained for the target organism. A recombinant DNA generator can autonomously create a recombinant DNA molecule by combining the fragment gene sequence with a vector DNA. Further, a host combining component can autonomously combine the recombinant DNA molecule with a host organism. The result can be a population of the host organism comprising the fragment gene sequence in the recombinant DNA molecule. Additionally, a recombinant DNA molecule harvesting component can autonomously harvest the recombinant DNA molecule, comprising the fragment gene sequence, from the population of the host organisms. This may result in in a plurality of cloned DNA molecules comprising the fragment gene sequence.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

FIG. 1 is a flow diagram illustrating an example process flow where one or more portions of one or more techniques and systems described herein may be implemented.

FIG. 2 is a flow diagram illustrating an example method where one or more portions of one or more techniques and systems described herein may be implemented.

FIG. 3 is a component diagram illustrating an example implementation of one or more portions of one or more systems described herein.

FIG. 4 is a component diagram illustrating an example implementation of one or more portions of one or more systems described herein.

FIG. 5 is a component diagram illustrating an example implementation of system for cloned genes sequences for plant characteristic enhancement.

FIG. 6 is a component diagram illustrating an alternate implementation of system comprising one or more portions of one or more systems described herein.

FIG. 7 is a flow diagram illustrating an example process flow of a method for cloning genes sequences for plant characteristic enhancement.

FIG. 8 is a flow diagram illustrating an example process flow where one or more portions of one or more techniques and systems described herein may be implemented.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

In one aspect, Organic Genome Enhancement (OGE) technology can utilize a novel process for collecting recurrently segmented organic nucleotide replications (e.g., targeted DNA or RNA sequences). In this aspect, these recurrently segmented organic nucleotide replications can be produced and processed in fully automated, computer controlled, USDA Certified Organic, CGMP Certified production system. Further the resulting recurrently segmented organic nucleotide replications can be cloned into recombinant molecules that are capable of organically producing precision edits of inhibiting traits in the genome of plants, animals, or other organisms, to enhance their natural biological processes, for example.

In this aspect, an OGE fully automated computer controlled production system can provide geneticists and plant breeders precision control of a culture, and utilization of collections of organically produced and processed DNA sequences that area cloned in unicellular organism. In one implementation, the sequences can contain specific organic segments of DNA that are utilized to generate precision edits to provide improvements to the genome of the target organism.

In one implementation, in this aspect, the fully automated, computer controlled production OGE system can precisely culture organic nucleic acid polymers deoxyribonucleic acid (DNA) and ribonucleic acid (RNA), composed of three subunit molecules: a nitrogenous base, a five-carbon sugar (ribose or deoxyribose), and at least one phosphate group. In this implementation, the fully automated, computer controlled production OGE system can carry nucleoside triphosphates ATP, GTP, CTP and UTP, unicell synthesizing amino acids, including proteins, cell membranes, and other fragments, that are created to control the movement of the unicell both internally and intercellularly. Further, in this implementation, an example OGE system can produce cell signaling pathways (e.g., cAMP-dependent pathway aka adenylyl cyclase, and cGMP signaling pathway), and can be incorporated into important cofactors of enzymatic reactions (e.g. coenzyme A, FAD, FMN, NAD, and NADP+).

As an example, the fully automated computer controlled OGE systems described herein, and used for genome editing, has many potential applications, including medicine and crop seed enhancement. The systems described herein may be used for organic molecular cloning, for organically assembling recombinant DNA molecules and directing their replication within host organisms. For example, the use of the word cloning can refer to the method involving the replication of one nucleic acid molecule to produce a population of cells with identical nucleic acid molecules.

In one implementation, the example OGE molecular cloning technology can use DNA sequences from two different organisms: the species that is the source of the DNA to be cloned, and the species that will serve as the living host for replication of the recombinant DNA. In this implementation, the DNA to be cloned is obtained from the organic organism of interest, and then treated with organic enzymes in a computer controlled environment to generate smaller organic DNA fragments. Further, these fragments can combine with organic vector DNA to generate organic recombinant DNA molecules. Additionally, the organic recombinant DNA can be introduced into an organic host organism.

For example, this can result in a population of organic unicell organisms in which recombinant DNA molecules are replicated along with the host DNA. This process can takes advantage of the ability of a single unicell being induced to precisely edit a single recombinant DNA molecule. For example, this single unicell can then be propagated exponentially, where each of the propagated cells contain copies of the original recombinant molecule. Thus, both the resulting population and the recombinant DNA molecule may be commonly referred to as “clones”. In one implementation, the resulting organic DNA sequences can be inserted into organic plasmids that are carried and digested to produce a desired genome enhancing edit to the host organism.

As one example, a similar but different CRISPR genome editing technology may be described as “Segments of prokaryotic DNA containing short, repetitive base sequences. As an example, in a palindromic repeat, respective repetition can be followed by short segments of spacer DNA from previous exposures to foreign DNA. Further, in this example, a version of the CRISPR/Cas system, CRISPR/Cas9, may successfully edit the genomes or organisms by delivering the Cas9nuclease complexed with a synthetic guide RNA (e.g., gRNA) into a cell. The cell's genome can be cut at a desired location, allowing existing genes to be removed and/or new ones added. Additionally, for example the similar but different CRISPR Cas9-gRNA complex corresponds with the CAS III crRNA complex, and at may provide illustrate that the Organic Genome Enhancement (OGE), fully automated computer control systems, can provide a successful precision control of the culture and utilization of collections of organically produced and processed DNA sequences. In one implementation, these DNA sequences may be cloned in unicellular organism and used to make precision edits that provide exhibitable improvements to a genome of food crop organisms, for example, that may still be marketed and sold as an “Organic Non-GMO” product.

Plant propagation often involves growing new plants in a controlled environment. These may involve genetically altered or may be plants that many copies all exactly alike are desired. Nearly all outcomes can be accomplished through tissue culture of plant cells or small tissue pieces from the plant of interest. These small pieces may come from a single mother plant or they may be the result of genetic transformation of single plant cells which under precise laboratory conditions are nurtured to grow and ultimately develop into a whole plant. Tissue culture techniques are often used for commercial production of plants as well as for plant research.

Tissue culture involves the use of small pieces of plant tissue e.g. plantlets or explants which are cultured in a nutrient medium under sterile conditions. Using the appropriate growing conditions for each explant type, plants can be induced to rapidly produce new shoots, and, with the addition of suitable hormones new roots. These plantlets can also be divided, usually at the shoot stage, to produce large numbers of new plantlets. Green shoots are generally observable within three weeks, and roots develop within six weeks. The new plants can then be placed in soil or soilless media to be grown in the normal agriculture methods (5).

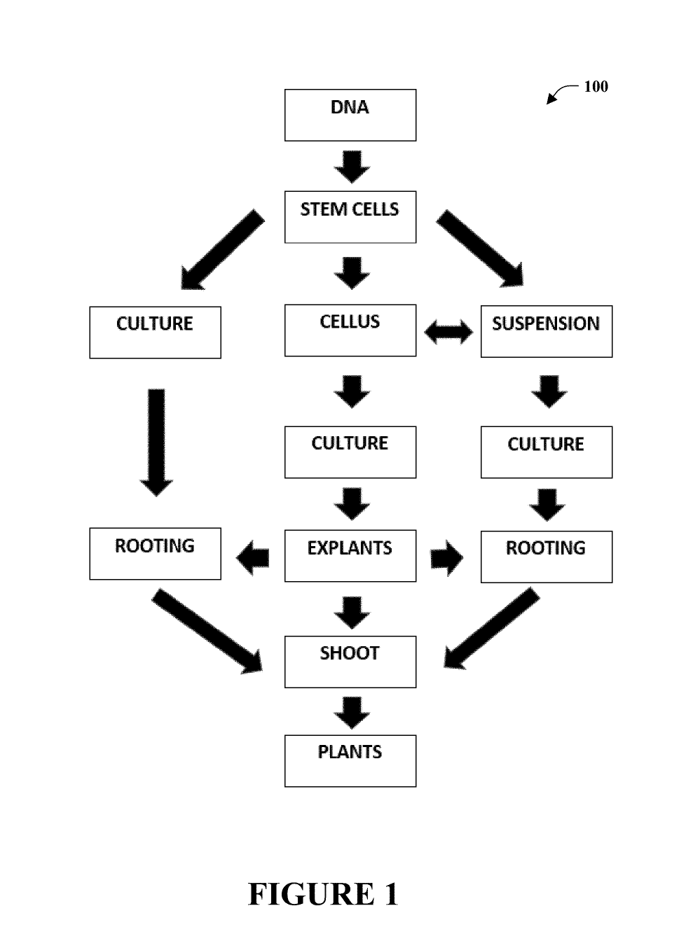

Micropropagation Organogenesis Technology is a very versatile and evolved method of tissue culture. It can take place by direct or indirect methods (e.g., see diagram 100 illustrated, in FIG. 1). This method utilizes skilled plant scientists and technicians, well equipped biological laboratory, and much preparation and effort to successfully propagate into full rooted plant.

Systems and techniques, disclosed herein, provide for genomics technology that may enable one to digitally sequence the genomes of the most desirable plant varieties, for example, based on flavor and nutrition. As an example, these desirable traits can be used to improve the most desirable commercial varieties with traits like increased yield, and quality, thus enhancing consumer demand for the product and increasing profitability.

In one implementation, a proprietary digital genetic database can be developed and used as an online cloud based library of digitally sequenced genomes and phenotypic (e.g., chemical composition) information on existing and new plant varieties. In this implementation, digital plant cloning technology disclosed herein can enable geneticists to select desired plant varieties to hybridize, improving and increasing crop quality and yields, by combining desirable genetics, thereby improving the variation of positive inherited characteristics in respective varieties.

One implementation comprises an apparatus for controlled environment plant growth, which uses automatic controls, and starts with plant cells, plantlet tissue, and/or plant cell clone propagation, to grow into whole plant crops. The devices and methods, disclosed herein, are easy to use, automated, and can replace many laboratory tools and techniques typically performed by a skilled biological technician. As a result, whole plant crops can be grown that exhibit advantageous traits and can be grown economically in large numbers for large scale research or commercial production.

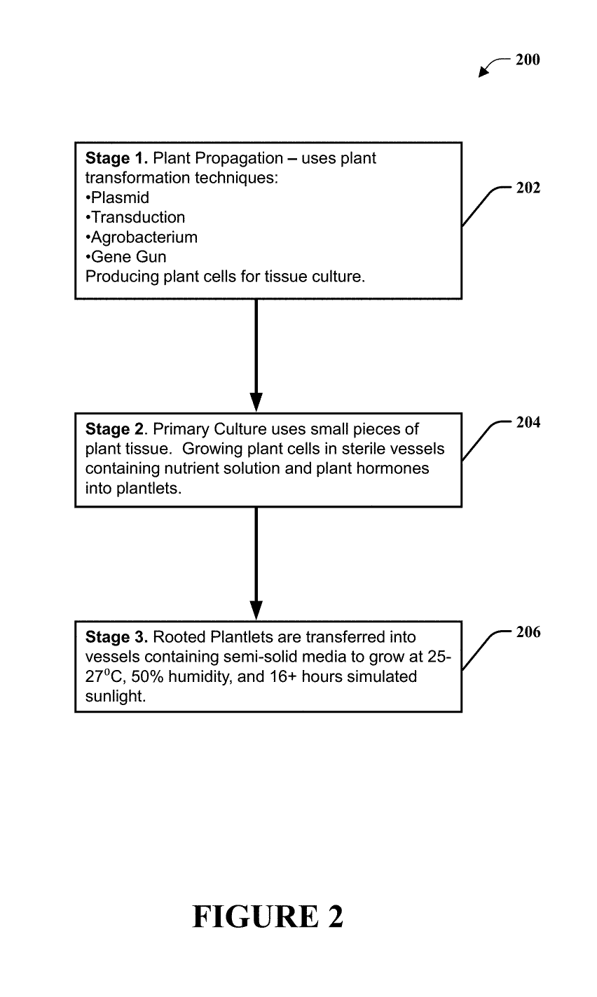

FIG. 2 is a flow diagram illustrating an exemplary method 200 controlled environment plant selection, propagation and growth, among other things. In one implementation, the method can comprise three stages.

Stage 1. Plant Propagation or Transformation Techniques 202—involves several direct or indirect methods in transforming plant cells. Some of the indirect methods used to transform plant genetic code:

Plasmid

A genetic structure in a cell that can replicate independently of the chromosomes, typically a small circular DNA strand in the cytoplasm of a bacterium or protozoan. Plasmids are much used in the laboratory manipulation of genes.

Transduction

Transduction is the process by which a gene or foreign DNA is transferred into an organism (e.g. plants, mammalian cells) cell by viral vector[7]. Transduction is a common tools used by molecular biologists to stably introduce a foreign gene into host cell's genome.

Agrobacterium Transformation

Agrobacterium tumefacience mediated transformation include the following steps:

The plasmid is removed from bacterium and the T-DNA is cut by restriction enzymes. Foreign DNA or gene is cut by the same restriction enzyme and inserted into the T-DNA of the plasmid. The plasmid is reinstated into the bacterium and the bacterium is used to insert the T-DNA containing foreign DNA into the chromosome of plant cell. The plant cells are grown in culture. A plant is generated from a cell clone. All of its cells carry the foreign gene and may express a new trait.

Gene Gun Method of Plant Transformation

A gene gun or a biolistic particle delivery system, originally designed for plant transformation, is a device for delivering exogenous DNA (transgenes) to cells. The payload is an elemental particle of a heavy metal coated with DNA (typically plasmid DNA). This technique is often simply referred to as biolistics. The gene gun is a good example of a creative idea being developed into a practical technology. Microscopic gold particles are used as ‘bullets’ to deliver DNA into callus cells. The gold particles are coated with hundreds of copies of the gene of interest.

Stage 2. Primary Culture 204—using small pieces of plant tissue growing plant cells in sterile vessels containing nutrient solution and plant hormones into plantlets.

Stage 3. Rooted Plantlets 206—growing out to robust rooted plants

In one implementation the apparatus may utilize a cloud based digital technology, wireless controls, and automated hardware to control the environment programed to propagate cell cultured plant varieties for optimum growth rate, and vigor (e.g., such as Clipius Technology's, see below). Plant propagating and cultivating device environmental conditions are designed using controlled light interval cycles, tailored atmosphere e.g. controlled temperature, humidity, enriched level CO2, and exacting air velocity. Additionally, plant cell and root zone environment are controlled to precise hydration, nutrient concentration, and beneficial sterile or protected habitat for optimum plantlet and whole plant vitality.

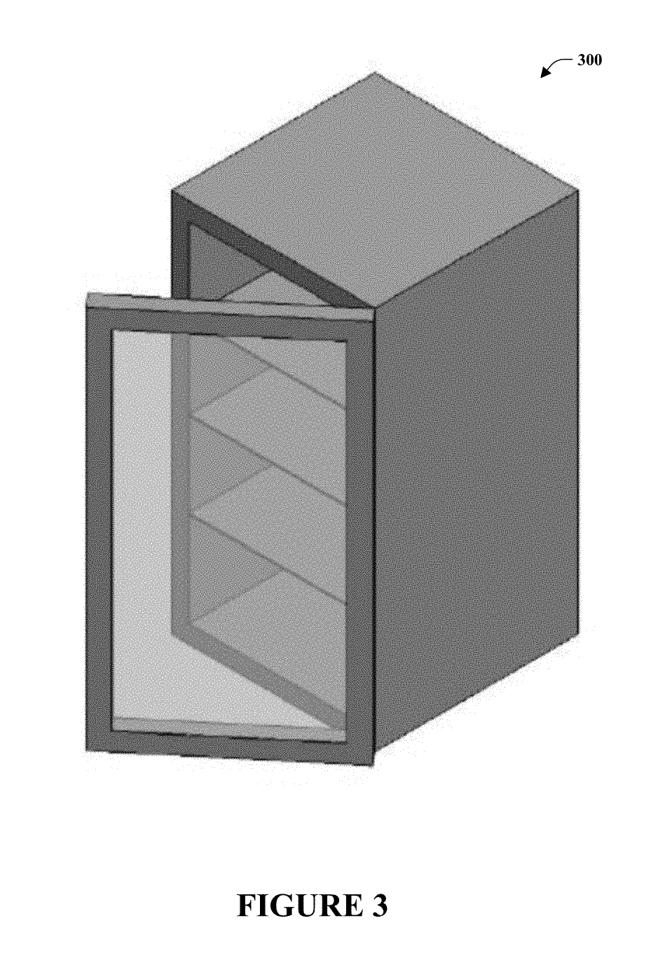

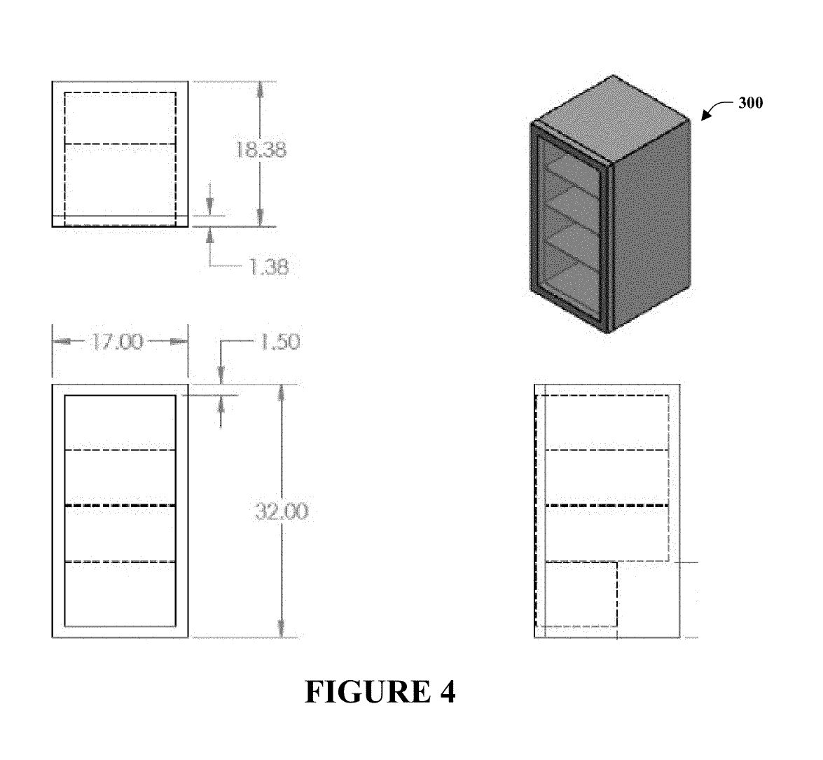

FIGS. 3 and 4 are component diagrams illustrating example implementations 300 of one or more portion of the systems described herein.

For example, Clipius Technology utilizes a device for monitoring critical parameters in the controlled environment applications, specifically temperature, humidity and light, and then controlling them through a microprocessor interface so as to create an optimal environment for growing plants, genomic applications, etc. Further, a computer application is used interacts with the physical parameters within the enclosure via a mobile interface. In this example, the device integrates a temperature sensor, a humidity sensor and a light sensor with a microprocessor. Additionally, a Bluetooth connection is used from the microprocessor to a computer or a mobile device, and via that to the cloud. The device also has algorithms to monitor the parameters sensed and based on the values control the compressor, a Peltier device or a light source so as to control the temperature, humidity and the light intensity.

In one implementation, transgenic plant crop digital gene sequencing and editing may be performed using a digital to biologic cloning platform (e.g., SGI-DNA digital synthetic DNA sequencer) within the incubation device and method disclosed herein. For example, the apparatus can genetically modify a plant species for desired trait(s). Further, in this example, the apparatus can process changes/edits made to gene (e.g., recombinant DNA) and/or desired gene to be transferred into targeted plant species' tissue/stem cells) or plasmid digitally, and can provide the means for efficient delivery of gene (s) to targeted cells for genetic modifications. Additionally, in this implementation, the device can convert genetic information or whole genome information/footprint of a plant species and convert the digital genomic footprint into a live plant. As an example, the technology may allow plant breeders to clone crops' genetic sequence, digitally record genetic information, store whole genome sequence information, and wirelessly transmit plant crops genomic data with agricultural desirable traits on demand in any geographical location. In this implementation, the digital plant genomic information can be synthetically transferred into viable biological cells, tissue, or clone bodies to propagate and grow into immature or mature plants holding commercial value.

In one implementation, genome information, such as a genetic sequence or footprint of a particular characteristic of a plant (e.g., related to plant yield, or taste, pest resistance, chemical resistance, height, color, etc.), may be identified and digitally uploaded to a computing system (e.g., cloud-based file management system). In this implementation, the genome information (e.g., the DNA code sequence) for the desired characteristic can be manipulated to create variations or alternate embodiments of the desired characteristic. Further, physical, synthetic versions of one or more of the alternate gene sequences may be digitally created in a digital gene sequencing portion of the systems disclosed herein. Additionally, respective alternate synthetic sequences can be inserted into plant cells or other plant material (e.g., automatically), and grown into resulting plants or plantlets, etc., in the automated incubation portion of the systems disclosed herein. As an example, the relatively automated and easy gene sequence creation, insertion into plant matter, and propagation of the resulting plants may allow for the alternate variations to be rapidly produced and exhibited in plants. In this example, the exhibited characteristics resulting from the alternate variations can be studied to identify whether they meet desired results.

In one aspect, a system can be devised for propagating cloned genes sequences for plant characteristic enhancement. For example, targeted gene sequences can comprise characteristics that may be desirable in a target plant (e.g., crop plant). That is for example, a first organism may exhibit a specific characteristic (e.g., latex production), and the target gene sequence that expresses that specific characteristic may be identified. Further, the target gene sequence may be extracted from the base organism or synthetically created from gene code for the base organism, duplicated, and inserted in to a target plant. In this example, the target plant can be propagated, and my exhibit the characteristic of the target gene sequence.

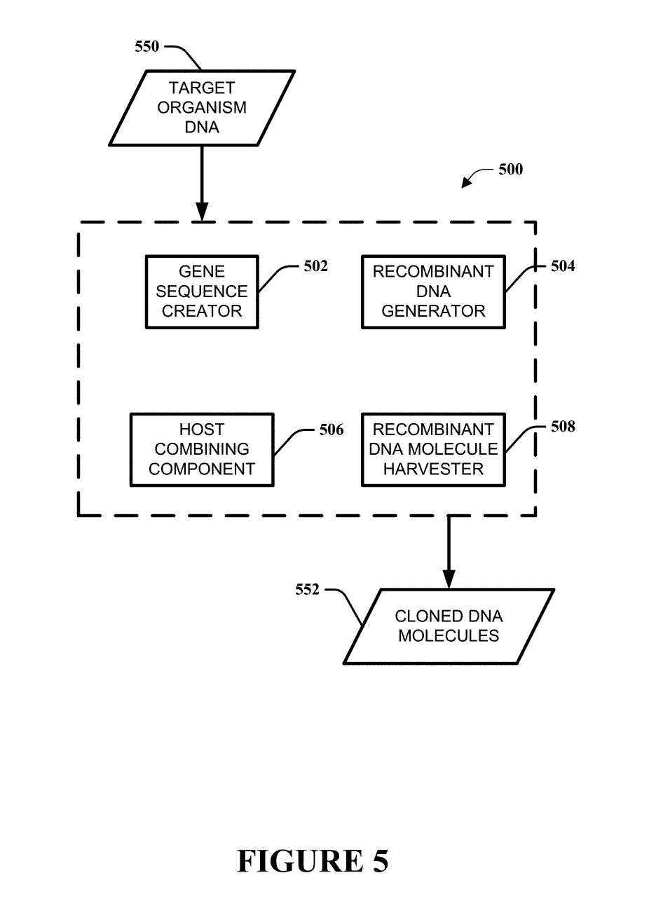

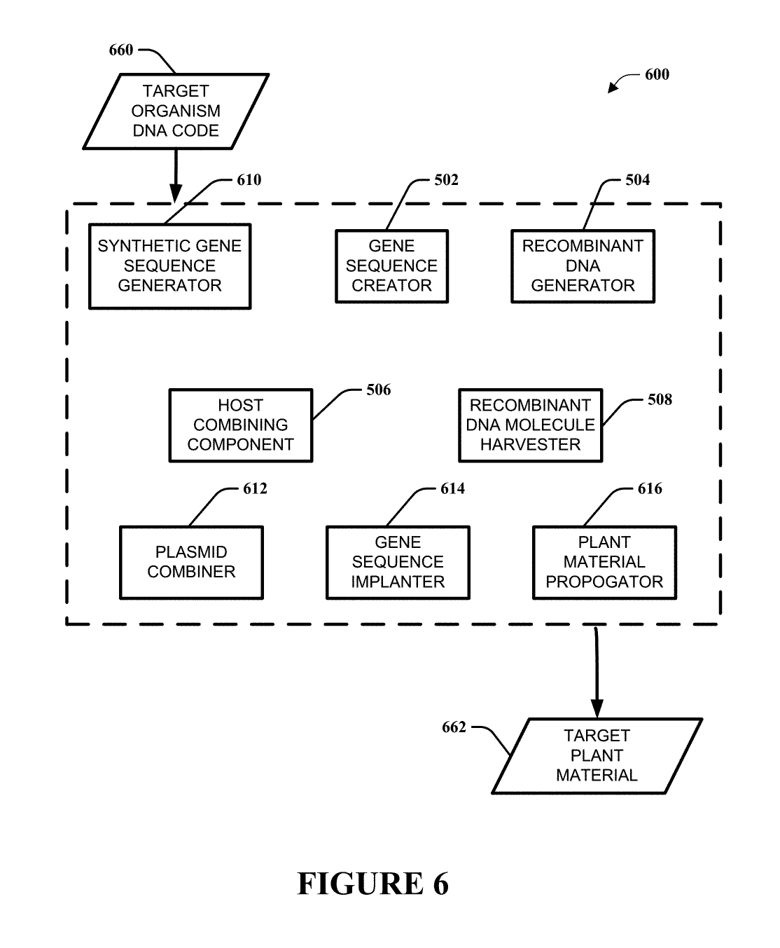

FIGS. 5 and 6 are schematic diagrams illustrating implementations of example systems 500, 600 for propagating cloned genes sequences for plant characteristic enhancement. In FIG. 5, the example system 500 comprises a gene sequence creator 502 that autonomously creates a fragment gene sequence (e.g., target gene sequence). The fragment gene sequence comprises a desired characteristic expression from a target organism, and is created from deoxynucleic acid 550 (DNA) obtained for the target organism. Further, a recombinant DNA generator 504 autonomously creates a recombinant DNA molecule by combining the fragment gene sequence with a vector DNA. Additionally, a host combining component 506 autonomously combines the recombinant DNA molecule with a host organism. In this implementation, this results in a population of the host organism comprising the fragment gene sequence in the recombinant DNA molecule. The example system 500 comprises a recombinant DNA molecule harvesting component 508 that autonomously harvests the recombinant DNA molecule comprising the fragment gene sequence from the population of the host organisms. In this implementation, this results in a plurality of cloned DNA molecules 552 comprising the fragment gene sequence.

In FIG. 6, an alternate example, system 600 further comprises a synthetic gene sequence generator 610. The synthetic gene sequence generator 610 autonomously generates the fragment gene sequence comprising the desired characteristic expression from the target organism. The synthetic gene sequence generator 610 uses a stored computer-based file indicative of the gene code 660 of the desired trait for the target organism to autonomously combining nucleoside triphosphates with a nitrogen base and a five-carbon sugar. In an alternate implementation, the DNA obtained for the target organism can comprise DNA harvested from the target organism.

In this example implementation, the system 600 can comprise a plasmid combining component 612 that autonomously inserts the cloned DNA molecules into plasmids. Further, the system 600 can comprise a gene sequence implantation component 614 that autonomously implants the fragment gene sequence into target plant material. The implantation of the fragment gene sequence into target plant material can comprise coupling target map RNA on respective tips of the gene sequence in a stem cell batch of the target plant material. Additionally, the system 600 can comprise a plant material propagation component 616. The plant material propagation component 616 can comprise a controlled growth environment that autonomously grows a target plant material into plant tissue 662 that can be introduced or grown into at least a portion of a matured plant.

In one implementation, the host combining component 506 can utilize single guide ribonucleic acid (sgRNA) to help couple the fragment gene sequence with the recombinant DNA. In another implementation, the host combining component can autonomously implant the fragment gene sequence into the recombinant DNA by coupling target map RNA on respective tips of the gene sequence in the stem cell batch. As an example, this can be used to unwind the DNA in the stem cell batch, and connect the tips of the fragment (e.g., target) gene sequence at a target location in the DNA. In this example, the DNA then reconnects and rewinds into a double helix. For example, using the sgRNA or target map RNA the fragment (target) gene sequence is specifically aligned to the right location in the chromosome to unwind and open; and subsequently connect the fragment with the target chromosome location.

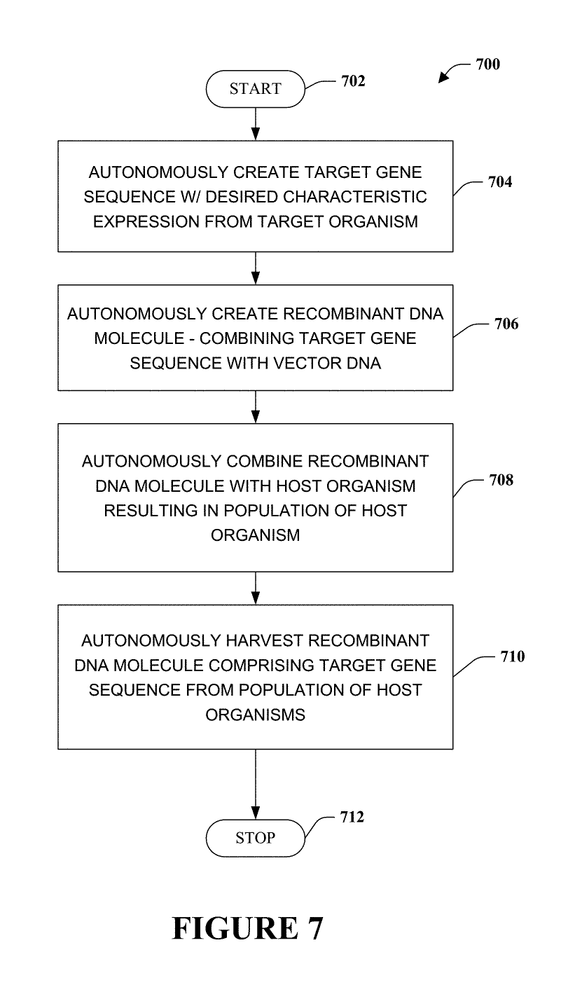

In one aspect, a method may be devised for propagating cloned genes sequences for plant characteristic enhancement. FIG. 7 is a flow diagram illustrating one example implementation of a method 700 for propagating cloned genes sequences for plant characteristic enhancement. The example method 700 begins at 702. At 704 a computer controlled system can be used to autonomously create a target gene sequence. The target (e.g., fragment) gene sequence comprises a desired characteristic expression from a target organism, from deoxynucleic acid (DNA) from a target organism. At 706, a recombinant DNA molecule is autonomously created by combining the target gene sequence with a vector DNA. At 708, the recombinant DNA molecule is autonomously combined with a host organism, resulting in a population of the host organism comprising the target gene sequence in the recombinant DNA molecule. At 710, the recombinant DNA molecule can be autonomously harvested. The harvest can comprise collecting the target gene sequence from the population of the host organisms, resulting in a plurality of cloned DNA molecules comprising the target gene sequence.

Having harvested a plurality of cloned DNA molecules comprising the target gene sequence, the example method 700 ends at 712.

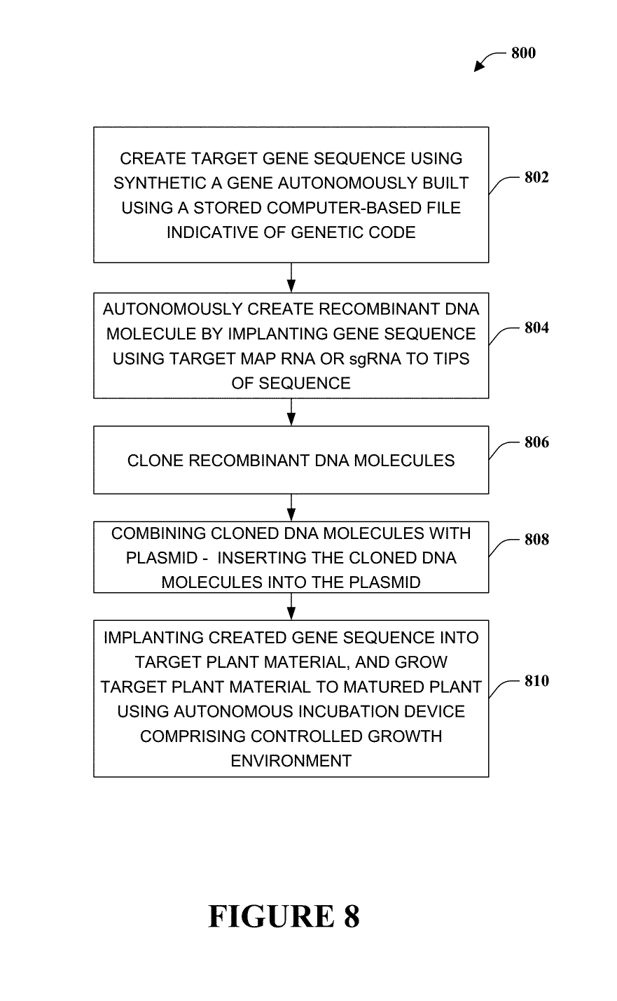

FIG. 8 is a flow diagram illustrating an alternate embodiment of an example method 800, where one of more portions of one or more techniques described herein may be implemented. At 802, a target gene sequence can be autonomously created. In this implementation, the creating the target gene sequence can comprise creating a synthetic gene sequence by autonomously building the gene sequence using a stored computer-based file indicative of genetic code identified as a portion of the target organism's genetic code that provides the desired characteristic. At 804, a recombinant DNA molecule can be autonomously created. This can comprise autonomously implanting the fragment gene sequence into the recombinant DNA by coupling target map RNA on respective tips of the target gene sequence.

At 806, the recombinant DNA molecule can be clones. For example, the recombinant DNA molecule can be autonomously combine with a host organism resulting in a population of the host organism comprising the target gene sequence in the recombinant DNA molecule. At 808, the cloned DNA molecules can be autonomously combed with a plasmid component by inserting the cloned DNA molecules into the plasmid. Further, at 810, the created gene sequence can be implanted into a target plant material. Additionally, the target plant material can be grown into at least a portion of a matured plant using an autonomous incubation device. The autonomous incubation device can comprise a controlled growth environment for the target plant material.

The word “exemplary” may be used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, At least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used in this application, the terms “component,” “module,” “system,” “interface,” and the like are generally intended to refer to a computer-related entity, either hardware, a combination of hardware and software, software, or software in execution. For example, a component may be, but is not limited to being, a process running on a processor, a processor, an object, an executable, a thread of execution, a program and/or a computer. By way of illustration, both an application running on a controller and the controller can be a component. One or more components may reside within a process and/or thread of execution and a component may be localized on one computer and/or distributed between two or more computers.

Furthermore, the claimed subject matter may be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device, carrier or media. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

For reference purposes, the following references are incorporated by reference into this disclosure:

1. U.S. Pat. No. 4,531,324A Plant Tissue Culture Device https://www.google.com/patents/US4531324#forward-citations

2. U.S. Pat. No. 5,597,731A Plant Propagation System https://www.google.com/patents/US5597731

3. U.S. Pat. No. 6,581,327 B2 Apparatus and Method for Propagating Plants https://www.google.com/patents/US6581327

4. CN 104936435A Plant Cultivating Device Using Artificial Light https://www.google.com/patents/CN104936435A?cl=en

5. Plant Tissue Culture Techniques, Roberta H. Smith; Academic Press 2000

6. Micro propagation: Technique, Factors, Applications, and Disadvantages, Nandkishor Jha http://www.biologydiscussion.com/biotechnology/clonal-propagation/micro-propagation-technique-factors-applications-and-disadvantages/10732

7. Transduction, Genetics at the US National Library of Medicine Medical Subject Headings (MeSH)

What is claimed is:

1. A system for propagating cloned genes sequences for plant characteristic enhancement, comprising:
a gene sequence creator that autonomously creates a fragment gene sequence, comprising a desired characteristic expression from a target organism, from deoxynucleic acid (DNA) obtained for the target organism;
a recombinant DNA generator that autonomously creates a recombinant DNA molecule by combining the fragment gene sequence with a vector DNA;
a host combining component that autonomously combines the recombinant DNA molecule with a host organism resulting in a population of the host organism comprising the fragment gene sequence in the recombinant DNA molecule;
a recombinant DNA molecule harvesting component that autonomously harvests the recombinant DNA molecule comprising the fragment gene sequence from the population of the host organisms, resulting in a plurality of cloned DNA molecules comprising the fragment gene sequence.

2. The system of claim 1, comprising a synthetic gene sequence generator that autonomously generates the fragment gene sequence comprising the desired characteristic expression from the target organism, by autonomously combining nucleoside triphosphates with a nitrogen base and a five-carbon sugar based on a stored computer-based file indicative of the gene code of the desired trait for the target organism.

3. The system of claim 1, the DNA obtained for the target organism comprising DNA harvested from the target organism.

4. The system of claim 1, the host combining component utilizing single guide ribonucleic acid (sgRNA) to help couple the fragment gene sequence with the recombinant DNA.

5. The system of claim 1, the host combining component autonomously implanting the fragment gene sequence into the recombinant DNA by coupling target map RNA on respective tips of the gene sequence in the stem cell batch.

6. The system of claim 1, comprising a plasmid combining component that autonomously inserts the cloned DNA molecules into plasmids.

7. The system of claim 1, comprising a gene sequence implantation component that autonomously implants the fragment gene sequence into target plant material, the implantation of the fragment gene sequence into target plant material comprising coupling target map RNA on respective tips of the gene sequence in a stem cell batch of the target plant material.

8. The system of claim 7, comprising a plant material propagation component, comprising a controlled growth environment, to autonomously grow a target plant material into plant tissue that can be introduced or grown into at least a portion of a matured plant.

9. A system for propagating plant matter comprising sequenced genes, comprising:
a gene sequence creator that autonomously creates a target gene sequence corresponding to one or more desired traits from a target organism, the target gene sequence generated from DNA from the target organism, resulting in a stem cell batch comprising the target gene sequence;
a gene sequence implantation component that autonomously implants the target gene sequence into target plant material, the implantation of the target gene sequence into target plant material comprising coupling target map RNA on respective tips of the gene sequence in the stem cell batch; and
a plant material propagation component, comprising a controlled growth environment, to automatically grow the target plant material into plant tissue that can be introduced or grown into at least a portion of a matured plant.

10. The system of claim 9, comprising a synthetic gene sequencing component that autonomously creates a synthetic version of the target gene sequence, resulting in a stem cell batch from the target organism, the code for the target gene sequence provided from a stored computer-based file indicative of the gene code of the one or more desired traits.

11. The system of claim 9, the gene sequence implantation component utilizing single guide ribonucleic acid (sgRNA) to couple the fragment gene sequence with the target plant material.

12. The system of claim 9, comprising a recombinant DNA generator that autonomously creates a recombinant DNA molecule by combining the target gene sequence with a vector DNA.

13. The system of claim 12, comprising a host combining component that autonomously combines the recombinant DNA molecule with a host organism resulting in a population of the host organism comprising the target gene sequence in the recombinant DNA molecule.

14. The system of claim 13, comprising a recombinant DNA molecule harvesting component that autonomously harvests the recombinant DNA molecule comprising the target gene sequence from the population of the host organisms, resulting in a plurality of cloned DNA molecules comprising the target gene sequence.

15. The system of claim 14, comprising a plasmid combining component that autonomously inserts the cloned DNA molecules into plasmids.

16. A method for propagating cloned genes sequences for plant characteristic enhancement, comprising using a computer controlled system to:
autonomously create a target gene sequence, comprising a desired characteristic expression from a target organism, from deoxynucleic acid (DNA) from a target organism;
autonomously creates a recombinant DNA molecule by combining the target gene sequence with a vector DNA;
autonomously combining the recombinant DNA molecule with a host organism resulting in a population of the host organism comprising the target gene sequence in the recombinant DNA molecule;
autonomously harvesting the recombinant DNA molecule comprising the target gene sequence from the population of the host organisms, resulting in a plurality of cloned DNA molecules comprising the target gene sequence.

17. The method of claim 16, autonomously creating a target gene sequence comprising creating a synthetic gene sequence by autonomously building the gene sequence using a stored computer-based file indicative of genetic code identified as a portion of the target organism's genetic code that provides the desired characteristic.

18. The method of claim 16, autonomously creating a recombinant DNA molecule comprising autonomously implanting the fragment gene sequence into the recombinant DNA by coupling target map RNA on respective tips of the target gene sequence.

19. The method of claim 16, comprising autonomously combining the cloned DNA molecules with a plasmid component by inserting the cloned DNA molecules into the plasmid.

20. The method of claim 16, comprising autonomously:
implanting the created gene sequence into a target plant material; and
growing the target plant material into at least a portion of a matured plant using an autonomous incubation device comprising a controlled growth environment for the target plant material.
